(19) United States
(12) Patent Application Publication
(10) Pub. No.: US 2013/0158375 A1
(43) Pub. Date: Jun. 20, 2013

(54) Alarm Processor for Detection of Adverse Hemodynamic Effects of Cardiac Arrhythmia

(71) Applicant: Lawrence A. Lynn, Columbus, OH (US)

(72) Inventor: Lawrence A. Lynn, Columbus, OH (US)

(21) Appl. No.: 13/769,150

(22) Filed: Feb. 15, 2013

Related U.S. Application Data
(63) Continuation of application No. 11/708,422, filed on Feb. 20, 2007, now abandoned.
Application No. 11/708,422 is a continuation-in-part of application No. 11/351,961, filed on Feb. 10, 2006.
Application No. 11/351,961 is a continuation-in-part of application No. 10/150,842, filed on May 17, 2002, now Pat. No. 7,758,503.
Application No. 10/150,842 is a continuation-in-part of application No. 10/150,582, filed on May 17, 2002, now Pat. No. 7,081,095.

(51) Int. Cl. A61B 5/0205; A61M 16/00; A61B 5/0472; A61B 5/04; A61B 5/1455; A61B 5/00

(52) U.S. Cl. CPC A61B 5/0205; A61B 5/14551; A61B 5/742; A61B 5/746; A61B 5/0472; A61B 5/04012; A61M 16/0057; A61M 16/0051; A61B 5/7475
USPC 600/324

(57) ABSTRACT

The disclosed embodiments relate to an apparatus and method for providing a warning. In one example, an apparatus includes a sensor, which is configured to be coupled to a body of a patient and to output a photoplethysmograph signal, which is indicative of pulse waveforms in the body. The apparatus also includes a processor, which is coupled to process the photoplethysmograph signal so as to identify sequential pulse waveforms in the signal, the processor detecting a cardiac arrhythmia based on identifying a shape feature of the pulse waveform occurring simultaneously with a change in rate or rhythm of the pulse waveforms or an electrocardiographic waveform, and to output a warning responsive to the simultaneous occurrence.

### CROSS-REFERENCE TO RELATED APPLICATIONS

This application is a continuation application of U.S. patent application Ser. No. 11/708,422, entitled “Maneuver-Based Plethysmographic Pulse Variation Detection System and Method,” filed Feb. 20, 2007, the disclosure of which is hereby incorporated by reference in its entirety for all purposes, and this application is a continuation-in-part of U.S. patent application Ser. No. 11/351,961, entitled “System and method for automatic detection of a plurality of SPO2 time series pattern types,” filed Feb. 10, 2006, the disclosure of which is hereby incorporated by reference in its entirety for all purposes, which is a continuation-in-part of U.S. patent application Ser. No. 10/150,842, entitled “Microprocessor System for the Analysis of Physiologic and Financial Datasets,” filed May 17, 2002,(now U.S. Pat. No. 7,758,503, issued Jul. 20, 2010) the disclosure of which is hereby incorporated by reference in its entirety for all purposes, and a continuation-in-part of U.S. application Ser. No. 10/150,582, entitled “Centralized hospital monitoring system for automatically detecting upper airway instability and for preventing and aborting adverse drug reactions,” filed May 17, 2002, (now U.S. Pat. No. 7,081,095, issued Jul. 25, 2006) the disclosures of which is hereby incorporated by reference in its entirety for all purposes, which claims the benefit of U.S. Provisional Application Serial No. 60/291,691 filed on May 17, 2001 and claims the benefit of U.S. Provisional Application Serial No. 60/291,687 filed on May 17, 2001, the disclosures of which are hereby incorporated by reference in their entirety for all purposes.

### FIELD OF THE INVENTION

This invention relates systems and methods for detecting and monitoring adverse disorders in clinical medicine.

### BACKGROUND AND SUMMARY OF THE INVENTION

Acute reductions in venous return are potential problems in hospitals, nursing homes and in the home environment. Actions which reduce venous return, particularly those which increase the intrathoracic pressure are common in the critical care unit. Many factors other than blood volume affect the respiratory variation of pulse pressure, cardiac output and heart rate. This is particularly true when a patient has a component of respiratory distress. Systems which detect the magnitude of respiratory variation in pulse pressure as a means for determining blood volume or venous return are unreliable in situations wherein the patient is experiencing a significant increase in respiratory effort. There is a need for a system which reliably detects a reduction in venous return or blood volume.

### BRIEF DESCRIPTION OF THE DRAWINGS

FIG. 1 is a block diagram of a system that is adapted to analyze data corresponding to variations in a plethysmographic pulse signal in accordance with an exemplary embodiment of the present invention; and

FIG. 2 is a process flow diagram illustrating a method of processing patient data in accordance with an exemplary embodiment of the present invention.

### DETAILED DESCRIPTION

An exemplary embodiment of the present invention detects a cardiovascular variation indicative of reduced venous return in timed relation to a maneuver in addition to or other than tidal breathing, which maneuver is known to reduce venous return, so that the timed relationship of the maneuver can be determined in relation to the induced cardiovascular variation to thereby better establish the presence of reduced venous return. An exemplary embodiment of the present invention comprises a venous return assessment system and method. Furthermore, exemplary embodiments of the present invention may comprise a system and method to identify a timed pattern of at least one fall in venous return to, for example, identify patients with more sustained patterns of blood pressure fall or with incomplete recovery after the fall. Accordingly, an exemplary reduced venous return detection system comprises a hemodynamic signal detector, such as a pulse oximeter, an input device for automatically or manually inputting an occurrence of a maneuver, such as adjusting peep or changing a parameter on a mechanical ventilator), and a processor for generating a time series of a hemodynamic signal (such as a plethysmographic pulse signal) and for outputting an indication based on both the maneuver and the time series. In one exemplary embodiment, the processor is programmed to determine at least one variation of the pulse signal (such as the systolic variation of the plethysmographic pulse), to output a time series of the variation and to detect a threshold and/or pattern of variation and to output an indication based on the detection. The variation of the plethysmographic pulse signal is one example of hemodynamic variation data that corresponds to a variation in intravascular hemodynamics of a patient. In another exemplary embodiment, the processor outputs a signal corresponding to at least one pleth waveform component prior to the maneuver (such as the amplitude of the pleth signal, for example, the average minimum of the pleth signal, the average maximum amplitude of the pleth signal, or a value indicative of a respiratory-related plethysmographic waveform variation). The processor then outputs the pattern or value indicative of at least one pleth waveform component after the maneuver and then compares the value or pattern prior to the maneuver with the value or pattern after the maneuver. The processor can determine and/or calculate the difference between the pre-maneuver and post maneuver values.

One exemplary embodiment of detecting reduced venous return according to an exemplary embodiment of the present invention comprises measuring at least one pleth waveform component, inputting the occurrence of a maneuver on a patient into a processor, measuring at least one pleth waveform component after the maneuver, comparing the pleth waveform component measured before the maneuver to the pleth waveform component after the maneuver. Another exemplary embodiment includes the acts of deriving a time series of a pleth waveform component, providing an indication of the time of at least one maneuver along the time series and outputting the time series. Another exemplary embodiment may include the act of comparing a pleth waveform pattern before a maneuver to the pleth waveform pattern after the maneuver.

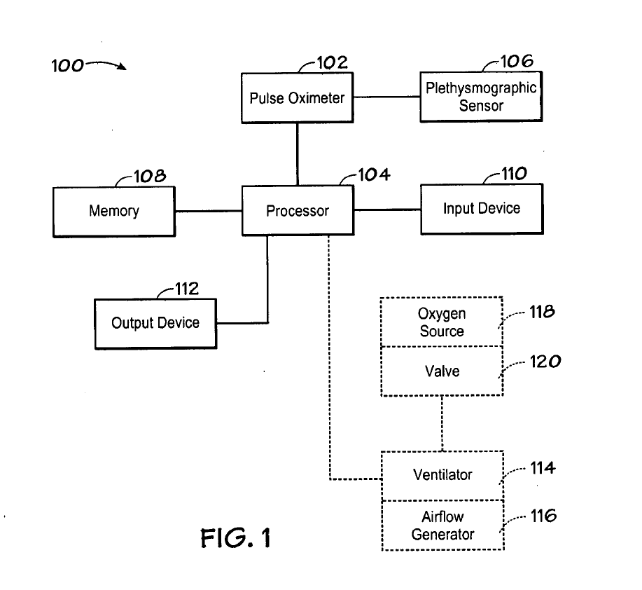

FIG. 1 is a block diagram of a system that is adapted to analyze data corresponding to variations in a plethysmographic pulse signal in accordance with an exemplary embodiment of the present invention. The system is generally referred to by the reference number 100. The system 100 comprises a pulse oximeter 102, which is connected to a processor 104. The processor 104 may be programmed to perform calculations and analysis on data corresponding to variations in a plethysmographic pulse signal. In the exemplary embodiment illustrated in FIG. 1, the pulse oximeter 102 is adapted to receive plethysmographic pulse data from a plethysmographic sensor 106, which may be connected to a patient. In an alternative embodiment, the processor 104 may be adapted to analyze previously obtained data stored in a memory 108, which is coupled to the processor 104. The exemplary system 100 may include an input device 110 to signal the performance of a maneuver by or on a patient. In this way, data being evaluated by the system 100 may be analyzed in the context of when it occurred relative to the performance of the maneuver. While an exemplary embodiment of the invention comprises the pulse oximeter 102, other devices that detect and/or monitor a hemodynamic pulse related parameter such as, for example, a pressure transduced arterial catheter, a continuous blood pressure monitor, or a digital volumetric plethysmograph, to name a few, may be employed to detect the hemodynamic and systolic pressure variations discussed below. The system 100 may additionally include an output device 112, such as a printer, display device, alarm or the like. The output device 112 may be adapted to signal or provide an indication of a condition detected by the processor 104.

Those of ordinary skill in the art appreciate that the detection and quantification of at least one pleth waveform component (such as magnitude of the respiratory related variation of the pleth) is possible. One method of processing the pleth signal is described in U.S. Pat. No. 7,081,095 (the contents of which are incorporated by reference as if completely disclosed herein). An example of a pleth waveform component is the pleth variation associated with ventilation as calculated from the plethysmographic pulse of the pulse oximeter 102, which is a sensitive indicator of intravascular blood volume in patients undergoing mechanical ventilation. The plethysmographic waveform (or pulse) variation can, for example, be outputted as a percentage of the peak pleth amplitude (see, for example, Pulse Oximetry Plethysmographic Waveform During Changes in Blood Volume, British Journal of Anesthesia, 82 (2): 178-81 (1999), the contents of which are hereby incorporated by reference as if completely disclosed herein).

However, while a decrease in effective venous return (as induced by a decrease in blood volume) commonly increases the respiratory-related pleth waveform (or systolic pressure) variation, a rise in respiratory effort can also increase this variation so that the linkage of this variation to the intravascular volume becomes much more complex in spontaneously breathing patients. Simplistic approaches, which attempt to determine the trend of the this plethysmographic waveform variation to determine blood volume, can provide a false trend which may suggest a falling blood volume due to a plethysmographic waveform variation cased by a rising respiratory effort due to bronchospasm, pulmonary embolism, or even an excess in blood volume inducing pulmonary edema.

The inventor of the present invention has recognized that, because the pleth waveform variation increases with both a fall in effective venous return or an increase in respiratory effort (which can be associated with excess venous return, heart failure and increases in lung water), the pattern of the pleth waveform variation (or other pleth waveform components) are best analyzed in timed relation to a maneuver (such as a change in a mechanical ventilation setting), which is known to reduce venous return, especially in disease states and in the presence of certain medications or in states of low blood volume so that the relationship of the change in pleth waveform variation to the maneuver can be determined to thereby better establish the presence of reduced venous return and to identify when the magnitude of venous return and/or the vasoconstrictive arterial response to a decline in venous return, is abnormal.

In an exemplary embodiment of the present invention, the processor 104 is programmed to detect a falling SPO2 combined with a rising magnitude of the pleth respiratory variation or a change or a pattern of change in a plethysmographic pulse component in relation to a maneuver that potentially reduces venous return. In an exemplary embodiment of the present invention, the processor 104 can be programmed, as by using an objectification method, to convert the plethysmographic time series into program objects such as dipoles (see, e.g. U.S. patent application Ser. No. 10/150,842 filed on Aug. 21, 2003 (now U.S. Patent Publication No. 20030158466), the contents of which are incorporated by reference as if completely disclosed herein) and objects comprised of events such as rises and falls and reciprocations (fundamental level).

Reciprocation objects can be defined by the user or by adaptive processing, as a threshold or pattern of reduction of amplitude, peak value, nadir value, slope, area under the curve (AUC) or the like. The components of the rises and falls such as the peaks, the nadirs, the slopes, or the AUC, to name a few, can be applied to render the composite level of the plethysmographic time series. The pattern of the reciprocations of one or more of these values (the composite level) can use used to detect respiration rate wherein the respiration rate is defined as the average number of reciprocations at the composite level per minute. More complex variations in the pattern of the plethysmographic pulse will also be detectable at the composite level such as apneas or sustained variations in blood flow to the finger (as, for example, may be induced by a mechanical ventilator setting change or a change in body position from the supine to the upright position). The SPO2 can be similarly processed in parallel with the pulse and the pattern of the pulse at the any level of the pulse compared with the pattern of the SPO2 at any level.

In an exemplary embodiment of the present invention, the number of reciprocations per minute and/or the magnitude of the amplitude of the reciprocations, amplitude, as determined by calculating the number of reciprocations per minute, is compared using the processor 104 with the time series of the SPO2 at, for example, the raw, dipole or fundamental level. The relationship between these two time series determined by the processor 104 may be used to detect and quantify the relationship between the ventilation time series (derived of the plethysmographic pulse) and the oxygen saturation time series.

In an exemplary embodiment of the present invention, the processor 104 is programmed to detect a change (such as a fall) in a plethysmographic pulse component (as for example the components noted above) in response to a maneuver, which affects venous return to the heart. Examples of such maneuvers include changes in a mechanical ventilator (such as an increase in positive pressure delivery to the patient, an increase in positive and expiratory pressure delivery to the patient, a change or changes in tidal volume, PEEP, respiration rate, I:E ratio, an exogenous ventilation maneuver, to name a few examples). The processor 104 can be programmed to automatically detect the maneuver or to receive an input from the input device 110 indicative of the occurrence or pattern of the maneuver. In an exemplary embodiment of the present invention, the input device 110 can be accessed through a menu which can allow the user to specify the maneuver.

In an exemplary embodiment of the present invention, the processor 104 is adapted to detect reduced venous return. An input is provided via the input device 110 when the patient undergoes a maneuver. The beginning of the maneuver may be taken into account when analyzing the corresponding SPO2, respiration and ventilation data. A variation in a least one component of the plethysmographic pulse may be quantified and a relationship between the variation and the maneuver may be identified. By way of example, a fall in the average pleth amplitude (such as the systolic variation) of about 20% or more in response to a maneuver can result in an output that indicates to an attendant that there is a potentially significant reduction in venous return in association with the maneuver. Alternatively, the processor 104 can be programmed to detect an increase in the reciprocation amplitude at the composite level of about 20-40% or more can output an indication of the presence and/or magnitude and/or pattern of orthostatic variation in the pleth amplitude pattern. In one exemplary embodiment of the present invention, the pulse oximeter 102 is adapted to be used for spot checks of the SPO2. The system may also be adapted to display a menu on, for example, either the input device 110 or the output device 112 depending on system design considerations. A user may specify that one or more maneuver(s) is (are) to be initiated via the menu. The user may then be instructed to press a button or touch the screen at the time the maneuver is initiated. The processor 104 tracks the pattern of the pleth and outputs and detects threshold pattern changes or lack thereof as noted above. An indication (such as a textual indication or alarm) of the presence or absence of threshold maneuver induced variation value and/or pattern may be provided. In addition, the slope or other components of the pattern of the variation subsequent to the maneuver can be determined and quantified. A time series indicative of the variation with the points of the occurrence of the maneuver marked along the time series may be outputted for over reading by the physician. Furthermore, a time series of one or more of the maneuvers may also be created. A time series of pleth variation data may be compared to the time series of one or more maneuvers.

In another exemplary embodiment of the present invention, the plethysmographic monitor system 100 serves as a pulse rate and pattern detection system. The processor 104 is programmed to determine the time intervals of the pleth including the time between pulses, and the time of systole, the time of diastole, the time of the rise, the time of the fall, and the pattern of pulses. Different patterns can be detected such as the pattern of atrial fibrillation (for example, identified by detecting an irregularly irregular interval between pulses and/or an irregularly irregular pulse amplitude), or a paroxysmal tachycardia (for example, detected by noting a precipitous increase in pulse rate which resolves precipitously). This pulse rhythm and pulse amplitude diagnostic function is complementary to the detection of a fall in venous return. This allows a routine ambulatory pulse oximeter to serve as a cardiac arrhythmia screener with the detection of premature beats (as well as the fall in pulse amplitude associated with premature beats to be detected and quantified. The presence of a severe fall in amplitude (for example 50% or more) suggests poor cardiac function or the presence of a ventricular premature beat. A high degree of pleth amplitude variation in a patient during routine rest monitoring, with a pattern which is not suggestive of atrial fibrillation is suggestive of significant cardiac disease. In one embodiment the magnitude of beat to beat variation of at least one component of the pleth (such as magnitude of variation of the pulse pressure) is determined and a time series of the variation is derived. The average and median variation for different time intervals is determined as a marker of cardiac function and health. If desired the variation can be filtered to eliminate or separate the cyclic variation which occurs with ventilation in some patients and both ventilation related variation and non ventilation related ventilation can be reported separately.

In yet another exemplary embodiment of the present invention, a time series of the respiratory rate (as for example determined from the pleth), a time series of the pleth variation, and a time series of the SPO2 are compared to identify the pattern relationships between these parameters such as a rise in pleth variation and a fall in SPO2, a rise in pleth variation and rise in respiratory rate, and/or a rise in respiratory rate and a fall in SPO2 and /or in relation to a maneuver. The processor 104 may be programmed to detect pathophysiologic divergence of the respiratory rate and/or the pleth variation and/or the SPO2.

In an exemplary embodiment of the present invention, an associated processor may be programmed to detect an oxygen saturation parameter (such as the ratio of ratios and/or the SPO2) and a respiration parameter (such as the respiration rate) and a magnitude of pleth variation. For example, the magnitude of pleth variation may be determined by the pleth amplitude and/or pleth slope variation. The pattern of the time series of the respiratory rate may then be compared with the pattern of the SPO2 to detect and abnormal relationship, such as pathophysiologic divergence with an increasing difference between the respiratory rate and the SPO2, for example. The processor may be programmed to output an indication based on the detection of the pattern or absolute value of the relationship and/or to output an index value indicative the relationship. The detection of a rise in respiration rate associated with a fall in plethysmographic pulse variation can be detected, quantified, and the pattern of the relationship analyzed and tracked by the processor. The processor can be programmed to provide an updated indication of the relationship and the pattern of the relationship to the user. The method of processing can, for example, be of the type discussed in U.S. Patent No. 7,081,095 (the contents of which is incorporated by reference as if completely disclosed herein). In an exemplary embodiment of the present invention, a plurality of parameters are combined to determine the global respiratory variation, including the amplitude of the events (at the fundamental level), the variation of the peak values (fundamental level), and the variation of the nadirs (also fundamental level).

The system 100 may comprise an optional ventilator 114 operatively coupled to the processor 104. The ventilator 114 may comprise an airflow generator 116 that is adapted to deliver an airflow to a patient. The system 100 may optionally include an oxygen source 118, the application of which may be controlled by the processor 104 via an optional oxygen flow valve 120. The processor 104 may be programmed so that the time series of the systolic pleth variation (for example) is displayed on the output device 112 adjacent a time series of at least one ventilation parameter. The processor 104 can be programmed for example to detect a pattern or threshold increase in systolic pressure variation in relation to a ventilator change and to output an indication of the pattern or threshold increase to the operator.

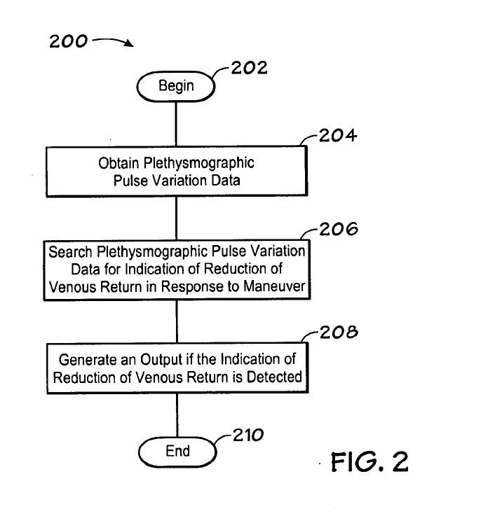

FIG. 2 is a process flow diagram illustrating a method of processing patient data in accordance with an exemplary embodiment of the present invention. The diagram is generally referred to by the reference number 200. At block 202, the process begins.

At block 204, plethysmographic pulse variation data is obtained. The plethysmographic pulse data, which corresponds to a variation in a plethysmographic pulse of a patient, may be obtained, for example, from a memory device or directly from monitoring a patient in real time. At block 206, the plethysmographic pulse variation data is searched for an indication of a reduction of venous return in response to a maneuver performed on or by the patient. An output, such as an alarm, printout and/or display, is generated if the indication of reduction of venous return is detected, as indicated at block 208. At block 210, the process ends.

In another embodiment the aforementioned time series objectification processing system can be employed with a plurality of parameters during a learning interval to automatically optimize subsequent therapy at subsequent times when less parameters are available for monitoring. In accordance with an exemplary embodiment of the present invention, during an initial learning period, at least one temporary target parameter is monitored in relation to the delivery of therapy in response to at least one working parameter. The target parameter is a parameter that is monitored temporarily during a learning period and that changes in relation to changes in the therapeutic parameter when those changes in the therapeutic parameter are made in response to a pattern or threshold value of a working parameter and wherein therapy applied in response to variations along the working parameter cause or would cause repeatable changes in the target parameter. While the working parameter provides desirable information concerning dosing or timing of the therapy, it may not be linearly or otherwise optimally related to the therapeutic goal so that it is generally the target parameter which is more completely indicative of the therapeutic goal.

According to an exemplary embodiment of the present invention, during a learning period the processor 104 (FIG. 1) recognizes at least one relationship between at least one characteristic of a time series of therapeutic parameter and at least one characteristic of a time series of a working parameter (which may be a preset relationship), and identifies a pattern or threshold value along the time series of the target parameter which is associated with that relationship. If the time series of the target parameter is not exhibiting the desired pattern or threshold value, the generated therapeutic output (and the associated the times series of the therapeutic parameter) is then repeatedly adjusted to change at least one of its characteristics in relation to the time series of the working parameter, until the desired pattern or threshold value along the time series of the target parameter is achieved. The relationships between the characteristics of the time series of the therapeutic parameter and characteristics of the time series of the working parameter which is associated with the desired pattern or threshold value in the target time series are termed “therapeutic characteristic matches” and are stored to memory. The step above can be repeated during the learning period for various ranges of breathing patterns and values (as by having the patient proceed through different maneuvers such as exercise, talking, or eating) to identify the “therapeutic match” for each range of breathing patterns and/or values.

During routine operation, after the learning period has been completed, the processor 104 (FIG. 1) is programmed to respond to dynamic changes in the time series of the working parameter by frequently adjusting therapy to maintain the presence of at least one of the therapeutic matches to achieve desired patterns and thresholds of the target parameter without the need to monitor the target parameter. If no match is available, the processor 104 (FIG. 1) adjusts the therapy to a default value. If a high number of adjustments to a default value are occurring, the processor 104 (FIG. 1) is programmed to notify the user that additional learning intervals may be useful.

In one exemplary embodiment, the target parameter is physiologically linked to the working parameter and can be the physiologic subordinate of the working parameter so that specific therapy applied in timed response to specific patterns or events along the working parameter will produce repeatable changes along the target parameter.

According to one aspect of the present invention, the automated detection of patterns or timing events along at least one time series of at least one working parameter is used to trigger delivery of a therapy while a target parameter is being monitored during a learning period and this timing is adjusted until the desired pattern(s) or threshold(s) of the target parameter is achieved. The timing and dose of therapy in relation to specific patterns or timing of events along at least one time series of at least one working parameter which achieved the desired time series of the target parameter is then recorded by the processor 104 (FIG. 1) and used for subsequent delivery of therapy when time series of the target parameter is not available. In one exemplary embodiment, an auto optimization algorithm is initially defined during at least one learning period with a plurality of target parameters.

An exemplary embodiment of the present invention comprises a processor-driven ambulatory oxygen conservation and therapy system. During ambulatory oxygen therapy, it is readily possible to continuously monitor nasal pressure through a nasal cannula but it is cumbersome to continuously monitor the SPO2. However, SPO2 is the target parameter that is preferably optimized during routine day to day activities, such as exercise and sleep. According to an exemplary embodiment of the present invention, the processor 104 (FIG. 1) can be programmed to control the output of an oxygen delivery device using an inputted time series of the SPO2 as a target parameter during a temporary learning period to identify desirable oxygen flow characteristics in response to specific breathing characteristics. In this embodiment, the SPO2 is applied as a target parameter and the nasal pressure is applied as a working parameter. Oxygen flow from the oxygen delivery system toward the cannula is applied as the therapeutic parameter. The processor 104 (FIG. 1) is programmed to control the valve 120 on the oxygen source 118 to deliver a specific pattern and/or rate of oxygen flow through the nasal cannula in relation to at least one specific pattern and/or rate of breathing, and to detect the occurrence of an unfavorable or favorable SPO2 pattern or value, and to adjust the oxygen flow characteristics upon the occurrence of an unfavorable SPO2 pattern or value until a desirable SPO2 pattern or value is identified. The processor 104 (FIG. 1) identifies the timing rate and pattern relationship between oxygen flow (the oxygen flow characteristics) and the timing rate and pattern of breathing (the breathing characteristics) which are associated with a favorable SPO2 pattern or value and thereby identifies a “therapeutic characteristic match”. The processor 104 (FIG. 1) is programmed to apply the therapeutic characteristic match during a subsequent routine operation period by adjusting to the matched oxygen flow characteristics whenever a given previously detected breathing characteristic is detected.

In one exemplary embodiment of the present invention, the processor 104 (FIG. 1)—based method of optimization of a target physiologic parameter comprises the steps of: (1) placing a medical device having a processor, a therapeutic output, and monitoring sources of at least two physiologic inputs in monitoring communication and therapeutic connection with a patient; (2) initiating a training period; (3) during the training period, monitoring a first input indicative of the target parameter and further monitoring a second input indicative of a surrogate parameter; (4) adjust the timing of the therapy in relation to the surrogate parameter to improve the target parameter; (5) identify at least one timing relationship between the therapy and the surrogate parameter which is associated with the desired pattern or threshold of the target parameter; and (6) after the training period, delivering therapy in accordance with the identified relationship to achieve the desired pattern or threshold of the target parameter without monitoring the target parameter.

The exemplary embodiment discussed above can be used to address an issue that occurs with home oxygen supplementation. Conventional oxygen reservoir systems often include oxygen conservation systems that detect breathing by nasal pressure and provide a pulse of oxygen during inspiration to conserve oxygen (by the avoidance of the provision of potentially wasted oxygen during exhalation). In one exemplary embodiment of the present invention, a portable oxygen concentrator is provided to continuously replace the oxygen in a small reservoir (which may be an elastomeric reservoir capable of containing pressurized oxygen of a small volume ,for example, a volume of about 100 ml of oxygen or less). As discussed below, the processor 104 (FIG. 1) controls the valve 120 (FIG. 1) to deliver oxygen with highly efficacious timing and flow characteristics so that the concentrator and an associated battery can have much less weight and be compact and still provide sufficient oxygen (for example a continuous output of only 0.5 liter per minute but delivered in a 0.25 second pulse delivered with a substantially square waveform at a flow rate of 4 liters minute). In conventional oxygen delivery systems, inspiration effort is often quite variable in response to different activities. Additionally, the transmission of the effort to the nasal cannula may be delayed by dynamic hyperinflation (auto peep) which has to be overcome before negative pressure is generated at the nostril. In these situations, an important component of the pulse of oxygen may be provided too late or not at all in various situations associated with alterations in the breathing rates or patterns (such as exercise, talking or eating). Since this “oxygen pulse timing failure” commonly occurs during exercise when oxygen is needed most to reduce dyspnea it is a significant issue. For this reason, oxygen conserving devices are often least useful during intervals when the patient has the greatest need.

U.S. Pat. No. 6,371,114, which is entitled “Control Device for Supplying Supplemental Respiratory Oxygen,” the disclosure of which is incorporated by reference as if completely disclosed herein, describes a control device for supplying supplemental oxygen using a pulse oximeter. However, an aspect of the system disclosed in U.S. Pat. No. 6,371,114 is the dependence of a closed loop device on continuous, or at least frequent, measurements of oxygen for optimal oxygen conservation. The inconvenience of being connected to even a simple wrist oximeter with a transmitter-based connection to the oxygen conservation valve system is not conducive to optimal long term ambulatory application outside the hospital. This issue has hampered widespread application of such devices. There has long been a need for an oxygen conservation delivery system and method which does not need continuous or near continuous oxygen measurements to provide for optimal oxygen delivery and conservation during a wide range of physiologic states including exercise. An exemplary embodiment of the present invention is directed to such a system and method.

An exemplary embodiment of the present invention comprises the oximeter (or other oxygen detecting device) 102 (FIG. 1), in communication with the processor 104 (FIG. 1) controlling the oxygen flow valve 120 (FIG. 1) mounted to the source of oxygen 118 (FIG. 1). The processor 104 (FIG. 1) is programmed to learn the oxygen flow characteristics which achieve the desired target SPO2 value during various training periods such as rest, exercise, eating, and in relation to specific respiratory patterns, rates and respiratory efforts. Oxygen flow characteristics include, for example, the magnitude of the oxygen flow rate, the oxygen flow rate waveform, and/or the timing of the oxygen flow waveform in relation to the inspiration or expiration waveform. The processor 104 (FIG. 1) is further programmed to retain in memory the favorable settings defined during the learning periods and to apply those setting in response to variations in nasal pressure during routine use when an oximeter is not available.

In an exemplary embodiment of the present invention, the pulse oximeter, the processor 104 (FIG. 1), and the oxygen valve system can be connected to a conventional system for delivery of nasal cannula oxygen. The processor 104 (FIG. 1) can be configured to detect and record the nasal pressure time series (the surrogate parameter) contemporaneous with the timed oxygen saturation time series (the target parameter). The processor is further programmed to auto adjust the output of the oxygen flow valve 120 (FIG. 1) during a range of training periods to allow auto optimization of oxygen delivery and conservation for application during routine use (without the subsequent need for the oximeter). In one embodiment the processor 104 (FIG. 1) has a setting for “routine operation” when the oximeter would be not routinely be connected, and a setting for “oxygen delivery training,” when the oximeter is connected to the patient and the processor 104 (FIG. 1). The mode of operation can be selected from a menu or the training setting can be automatically triggered by the detection of acceptable SPO2 time series input of a compatible pulse oximeter. The training setting is intended to allow the user, or healthcare worker, to regularly update the processor 104 (FIG. 1)-induced outputted oxygen delivery response patterns to the inputted nasal pressure time series.

In an exemplary embodiment of the present invention, the processor 104 (FIG. 1) is further programmed to adjust the operation of the oxygen flow valve 120 (FIG. 1) if the SPO2 time series exhibits adverse patterns (examples of adverse SPO2 patterns include a fall below threshold value, a fall toward a threshold value having a threshold slope, and a cluster pattern of SPO2 reciprocation indicative of Cheyenne-Stokes Respiration, to name a few). The processing system which converts time series patterns into objects for analysis, as discussed previously in this application, can be used for analyzing and detecting patterns along the SPO2 (target) time series and for analyzing and detecting patterns along the breathing (surrogate) time series (such as nasal pressure time series) and the oxygen delivery (therapeutic) time series for comparing the time series to detect a relationship between a pattern(s) or object(s) (such as a fall or rise along one time series in relation to a fall or rise in the other time series after adjusting for the expected delay between the time series. Types of breathing patterns detected include those previously discussed, such as rises and/or falls (and reciprocations) in the slope, amplitude, or duration of at least one component of the reciprocations along a time series of nasal tidal pressure, and/or a times series respiratory rate. Also, relationships between reciprocations, and/or rises and falls can be detected as previously discussed.

In an exemplary embodiment of the present invention, the processor 104 (FIG. 1) is programmed to identify the pattern(s) of breathing (as by the nasal pressure waveform) which preceded a pattern of SPO2 (such as a range of specific fall patterns) and to detect specific components or relationships of that breathing pattern. Potential adverse pattern objects of breathing relevant oxygen delivery include, for example, an increasing slope (more rapidly negative) or amplitude (more negative) of consecutive falls along the nasal pressure time series or a reduction in the duration of the falls. These detected patterns may indicate the potential for higher inspiration flow rates (which may dilute the inspired oxygen) or shorter inspiration time (limiting the time for inspiration).

Upon detection of a specific adverse pattern (relevant oxygen delivery) of breathing and upon detection of an adverse pattern along the SPO2 waveform indicating that oxygen delivery is not optimal, the processor 104 (FIG. 1) is programmed to cause the valve 120 (FIG. 1) to modify the oxygen delivery to improve the SPO2. For example, upon detection of a shortening of the inspiration time in association with a subsequent adverse SPO2 pattern, the processor 104 (FIG. 1) is programmed to adjust the timing of the oxygen pulse delivery (in relation to the patent's inspiration or expiration), the oxygen flow rate, and the oxygen flow/time waveform, in response to the target SPO2 time series. The processor 104 (FIG. 1) is programmed to adjust for the delay (as discussed previously) when it makes a determination of the detected response of the pulse oximeter to the adjustments in oxygen pulse timing, flow rate, flow waveform, or any other change in oxygen delivery.

In one exemplary embodiment, the pulse oximeter is connected with the processor 104 (FIG. 1), which is programmed to adjust the oxygen flow characteristics in response to the time series of breathing (e.g. nasal pressure) based on the output of the pulse oximeter. In an example, the processor 104 (FIG. 1) can be programmed to respond to a fall in SPO2 below 90% (or another preferred value) by shifting the onset of the oxygen pulse to an earlier timing in response to the onset of detected inspiration (for example 50-100 milliseconds). In some cases, this shift may mean that the oxygen pulse will now be anticipatory and initiated before the detected inspiration the relationship can be maintained however by measuring the rate of breathing or the time between the onset or end of expiration and the selected onset of the shifted pulse and then using the rate of breathing or the onset or end expiration relationship to trigger the oxygen pulse. To improve the SPO2, the oxygen flow characteristics can be modified in many ways. For example the oxygen pulse can be shifted (provided earlier or delayed) or prolonged. Additionally, the oxygen flow or pressure waveform can be modified, or any of these approaches can be combined. In an exemplary embodiment of the invention, the processor 104 (FIG. 1) is programmed to proceed through a sequence of changes to oxygen flow characteristics to achieve a target SPO2 for each change in breathing characteristic. For example, for an increase in respiration rate above 14 or a rapidly upwardly sloping respiration rate the processor 104 (FIG. 1) may adjust the oxygen flow characteristics first initiating an earlier oxygen pulse, then if this does not produce a satisfactory SPO2 (after the expected delay of 0.5-2 minutes, for example), prolonging the pulse, then if this does not produce a satisfactory SPO2 after the expected delay, modifying at least a portion of the oxygen flow waveform (for example increasing the instantaneous oxygen delivery flow rate in the initial portion of the wave or prolonging the duration of the peak instantaneous flow rate along the wave. Once satisfactory target SPO2 has been achieved for a given set of breathing character tics, the effective oxygen flow characteristics (and the timed relationship of these oxygen flow characteristics to the breathing characteristic), are recorded to the memory 108 (FIG. 1) by the processor 104 (FIG. 1) and used later during the “routine operation” to adjust oxygen flow characteristics in response to changes in the characteristics of breathing without the presence for a pulse oximeter. In an example, during routine operation, in response to detection of a respiration rate of 10 and an inspiration time of 1-2 seconds, the processor 104 (FIG. 1) responds as programmed during the prior learning period to cause the valve 120 (FIG. 1) to generate an oxygen pulse with a square waveform at 4 liters per minute for one second, whereas upon subsequent detection by the processor 104 (FIG. 1) of the breach of a threshold rise in respiration rate to 16 breaths per minute (or, in another example, a fall in inspiration time to less than one second) the processor 104 (FIG. 1) may now respond (as also programmed during the prior learning period) to cause the valve 120 (FIG. 1) to make an adjustment to generate an changed oxygen pulse of 0.75 second duration with a decelerating waveform with a peak flow rate of 8 liters per minute. In this example, these therapeutic choices are assumed to have been identified by the processor as adequate to achieve the desired target SPO2 during a prior learning period.

Another exemplary embodiment of the present invention, which may be useful for the treatment of sleep disordered breathing, comprises the pulse oximeter 102 (FIG. 1), the processor 104 (FIG. 1), a ventilator 114 (FIG. 1) and an airflow generator 116 (FIG. 1) (such as a CPAP or Bi-level non-invasive ventilator) connected to a system for delivery of gas to the nose and/or mouth. The system for delivery of gas may comprise the oxygen source 118 (FIG. 1) and the oxygen flow valve 120 (FIG. 1). The processor 104 (FIG. 1) can be configured to detect and record the pressure or flow time series (the working parameter) contemporaneous with the timed oxygen saturation time series (the target parameter). The processor 104 (FIG. 1) is further programmed to auto adjust the output of the flow valve 120 (FIG. 1) or airflow generator 116 (FIG. 1) during a range of training periods to allow auto optimization of gas delivery for application during routine use (without the subsequent need for the oximeter). In one exemplary embodiment, the processor 104 (FIG. 1) has a setting for “routine operation” when the oximeter 102 (FIG. 1) would not routinely be connected, and a setting for “oxygen delivery training,” when the oximeter 102 (FIG. 1) is connected to the patient and the processor 104 (FIG. 1). The operational mode can be selected from a menu or the training setting can be automatically triggered by the detection of acceptable SPO2 time series input of a compatible pulse oximeter. The training setting is intended to allow the user, or healthcare worker, to regularly update the processor 104 (FIG. 1) induced outputted gas delivery response patterns to the inputted pressure and/or flow time series.

In an exemplary embodiment of the invention, the processor 104 (FIG. 1) is further programmed to adjust the operation of the gas delivery valve and/or flow generator if the SPO2 time series exhibits adverse patterns (examples of adverse SPO2 patterns include; a fall below threshold value, a fall toward a threshold value having a threshold slope, and a cluster pattern of SPO2 reciprocations, to name a few). The processing system which converts time series patterns into objects for analysis, as discussed previously in this application, can be used for analyzing and detecting patterns along the SPO2 (target) time series and for analyzing and detecting patterns along the breathing time series (such as flow time series) and the gas delivery (therapeutic pressure) time series for comparing the times series to detect a relationship between a pattern(s) or object(s) (such as a fall or rise along one time series in relation to a fall or rise in the other time series after adjusting for the expected delay between the time series. Types of breathing patterns detected include those previously discussed, such as rises and/or falls (and reciprocations) in the slope, amplitude, or duration of at least one component of the reciprocations along a time series of pressure or flow, and/or a times series respiratory rate. Also, relationships between reciprocations, and/or rises and falls can be detected as previously discussed. In an example, the processor 104 (FIG. 1) is programmed to identify the pattern(s) of breathing (as by the pressure and/or flow waveform) which preceded a pattern of SPO2 (such as a range of specific fall patterns) and to detect specific components or relationships of that breathing pattern. Potential adverse pattern objects of breathing relevant to oxygen delivery include, for example, cluster of flow or pressure reciprocations indicative of clusters of apneas, a progressively falling tidal pressure or flow amplitude of consecutive breaths along the pressure or flow time series. The adverse patterns indicative of upper airway and ventilation instability have been extensively discussed herein.

Upon detection of a specific adverse pattern of breathing and/or upon detection of an adverse pattern along the SPO2 waveform indicating that oxygen delivery is not optimal, the processor 104 (FIG. 1) is programmed to cause the flow generator or valve modify the delivery of room air and/or oxygen to improve the SPO2 in specific response to the type of SPO2 pattern detected with or without consideration of the pattern of another signal such as a ventilation signal. For example, upon detection of a cluster of SPO2 reciprocations, the processor 104 (FIG. 1) can be programmed to adjust the magnitude of the end expiratory pressure delivery (EPAP). In another example, upon detection of a rising ventilation rate or other magnitude and a falling SPO2, the processor 104 (FIG. 1) can be programmed to initiate oxygen or increase the oxygen flow rate. In another example, upon detection of a falling ventilation rate or other magnitude and a falling SPO2 (indicative of hypoventilation), the processor 104 (FIG. 1) can be programmed to the inspiration pressure (IPAP), the spontaneous breathing rate, and/or convert to a mandatory breathing rate, the oxygen flow rate, and the oxygen flow/time waveform, in response to the target SPO2 time series. The processor 104 (FIG. 1) is programmed to adjust for the delay (as discussed previously) when it makes a determination of the detected response of the pulse oximeter to the adjustments in therapy.

In one exemplary embodiment, the processor 104 (FIG. 1) is programmed to provide a menu offering different testing modes. The testing modes can be, for example, of the types discussed above or as disclosed in U.S. patent application Ser. No. 11/351,961, entitled “System and Method for Automatic Detection of a Plurality of SPO2 Time Series,” the contents of which are incorporated by reference as if completely disclosed herein, or U.S. patent application Ser. No. 11/351,690, entitled “System and Method for the Detection of Physiologic Response to Stimulation,” the contents of which are incorporated by reference as if completely disclosed herein. Examples of different modes that may be employed include; a first mode for sleep testing, a second mode for exercise testing, a third mode for maneuver testing, to name a few. By selecting the mode, the operator causes a respective program to be engaged, which provides an analysis of the SPO2 time series and any additional time series provided based on the selected mode. In one example, the processor 104 (FIG. 1) is programmed to receive automatic or manual input at the onset of an event and the end of the event, such as exercise. The processor is further programmed to compare the time series of SPO2 and/or pleth or other output of the oximeter prior to the event, during the event and after the event. The processor provides an output based on the comparison. The output can comprise, for example the average SPO2 at rest prior to exercise, the lowest SPO2 with exercise, the slope of the fall in SPO2 with exercise, the slope of the rise in SPO2 after exercise, the time to return to resting levels after exercise to name a few. The oximeter 102 (FIG. 1) can be a compact, hand held or patient-mounted oximeter with memory. A GPS monitor or other activity monitor (not shown) may be added to the system to provide an input of a time series to the processor indicative of activity for comparison with the time series of SPO2 and/or pleth. or other time series.

In another exemplary embodiment, a time series of SPO2, sound, and chest impedance is provided by a combined audio sensor and chest wall impedance lead (not shown) for adhesive application to the chest. Additional leads with or without additional incorporated audio sensors can be applied to other regions of the chest to provide simultaneous or near simultaneous impedance and a plurality of sound time series outputs form a plurality of locations on the chest to the processor 104. The plurality of sound outputs can be used to localize airflow and detect regional airflow limitation or failure (as, for example, indicative of a pneumothorax or mucous plug. The processor 104 (FIG. 1) receives the impedance time series and the audio time series and compares the impedance time series to the audio time series to identify when the chest wall is moving without breath sounds thereby detecting airway obstruction. A detected cluster pattern of chest impedance variation combined with a detected cluster pattern from the audio sensor can, for example, be analyzed in a manner described in the aforementioned patents.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

What is claimed is:

1. An apparatus for providing a warning, comprising:
a sensor, which is configured to be coupled to a body of a patient and to output a photoplethysmograph signal, which is indicative of pulse waveforms in the body; and
a processor, which is coupled to process the photoplethysmograph signal so as to identify sequential pulse waveforms in the signal, the processor detecting a cardiac arrhythmia based on identifying a shape feature of the pulse waveform occurring simultaneously with a change in rate or rhythm of the pulse waveforms, and to output a warning responsive to the simultaneous occurrence.

2. The apparatus of claim 1, wherein the change in shape feature comprises at least one of a fall in the amplitude, an upstroke, or an area under a curve of the pulse waveforms.

3. The method of claim 1, wherein the change in shape feature comprises the occurrence of an irregular pattern of the amplitude, an upstroke, or an area under a curve of the pulse waveforms.

4. The apparatus of claim 1, wherein the processor is programmed to convert the signal into a time series of sequential objects.

5. The apparatus of claim 1, wherein the processor is programmed to generate a time series of sequential objects comprising sequential positive reciprocations indicative of the sequential pulse waveforms each sequential positive reciprocation being comprised of a rise object coupled to a fall object.

6. The apparatus of claim 5, wherein the detection comprises detecting a precipitous increase in frequency of the reciprocations associated with a fall in an amplitude, an area under the curve, or a slope of rise events of the reciprocations.

7. The apparatus of claim 1, wherein the detection comprises detecting sequential positive reciprocations comprised of a rise object coupled to fall object, the detection comprising detecting an irregular pattern of the reciprocations associated with a variable amplitude of the reciprocations.

8. The method of claim 1, wherein the cardiac arrhythmia comprises wide complex tachycardia.

9. The method of claim 1, further comprising providing a display of an indication of cardiac arrhythmia based at least in part on the determined cardiac arrhythmia.

10. An apparatus for providing a warning, comprising:
a sensor, which is configured to be coupled to a body of a patient and to output a photoplethysmograph signal, which is indicative of sequential blood pulses in the body; and
a processor, which is coupled to process the photoplethysmograph signal so as to identify at least one irregularity in a sequential pulse rhythm of the patient, to make a record indicating a time of occurrence of the at least one irregularity, and to process the record so as to provide a warning responsive to the occurrence.

11. The apparatus according to claim 10, wherein the pulses define photoplethysmographic peaks separated by peak to peak intervals, and photoplethysmographic areas under the pulses and the irregularity comprises a sudden decrease in the peak-to-peak intervals or increase in pulse rate in association with a sudden decrease in the photoplethysmographic areas.

12. An apparatus for providing a warning, comprising:
a sensor, which is configured to be coupled to a body of a patient and to output a photoplethysmograph signal, which is indicative of sequential blood pulses in the body; and
a processor, which is coupled to process the photoplethysmograph signal so as to generate a transformed signal based at least in part on a transformation of the photoplethysmograph signal, the processor being programmed to detect a change in a feature of the transformed signal; identify information indicative of a pulse rhythm abnormality based at least in part on the detected change in the feature of the transformed signal; and to identify the presence of a cardiac arrhythmia in a subject based at least in part on the information indicative of the pulse rhythm abnormality.

13. The apparatus of claim 12, wherein the transformation of the photoplethysmograph signal comprises a time series objectification.

14. The apparatus of claim 13, wherein the transformed signal comprises a time series of sequential objects.

15. The apparatus of claim 14, wherein the processor is programmed to generate a time series of sequential objects comprising sequential positive reciprocations indicative of the sequential pulse waveforms each sequential positive reciprocation being comprised of a rise object coupled to a fall object.

16. The apparatus of claim 15, wherein the time series of sequential objects comprises sequential positive reciprocations comprised of a rise object coupled to a fall object, and the detection comprises detecting a precipitous increase in frequency of the reciprocations associated with a fall in an amplitude, an area under a curve, or a slope of rise events of the reciprocations.

17. The apparatus of claim 13, wherein the time series of sequential objects comprise sequential positive reciprocations comprised of a rise object coupled to a fall object, the detection comprising detecting an irregular pattern of the reciprocations associated with a variable amplitude of the reciprocations.

18. An apparatus for providing a warning, comprising:
a sensor, which is configured to be coupled to a body of a patient and to output a photoplethysmograph signal, which is indicative of sequential pulse waveforms in the body; and
a processor programmed to detect a change in the sequential pulse waveforms indicative of a diminution in the pulse waveform occurring in association with a change in pulse rate and to output an warning responsive to the detected association.

19. The apparatus of claim 18, wherein the change in pulse rate occurs slowly.

20. The apparatus of claim 19 wherein the change in pulse rate occurs precipitously.

21. An apparatus for providing a warning, comprising:
a first sensor, which is configured to be coupled to a body of a patient and to output a photoplethysmograph signal, which is indicative of sequential pulse waveforms in the body;
a second sensor configured to be coupled to the body of a patient and to output an electrocardiograph signal generating QRS complexes; and
a processor programmed to compare the signals and to detect the association of a change in the electrocardiographic signal and a substantially simultaneous change in the pulse waveforms and to output an indication responsive to the association.

22. The apparatus of claim 21, wherein the occurrence comprises a diminution of pulse waveforms occurring in association with a change in the QRS complexes of the electrocardiographic signal.

23. The apparatus of claim 21 wherein the change in the QRS complexes comprises a widening of the QRS complexes.
